(19) United States
(12) Patent Application Publication
(10) Pub. No.: US 2012/0040448 A1
(43) Pub. Date: Feb. 16, 2012

(54) Microreactors With Connectors Sealed Thereon; Their Manufacturing

(76) Inventors: Sylvain Maxime F. Gremetz, Montereau Fault Yonne (FR); Jean-Marc Martin Gerard Jouanno, Painted Post, NY (US); Olivier Lobet, Mennecy (FR); Stephane Poissy, Numauce Bouel (FR); Ronan Tanguy, Grez Sur Loing (FR)

(21) Appl. No.: 13/266,333

(22) PCT Filed: Apr. 28, 2010
(86) PCT No.: PCT/US10/32742

§ 371 Date: Oct. 26, 2011

(30) Foreign Application Priority Data
Apr. 28, 2009 (EP) 09305368.4

(51) Int. Cl. C12M 1/00; F15D 1/00; B32B 37/14; B01J 14/00

(52) U.S. Cl. 435/289.1; 422/240; 422/603; 137/561 R; 156/60

(57) ABSTRACT

The present invention deals with microfluidic devices (200) including a microreactor (20) and at least one connector (101) sealed thereon. It also deals with a method for manufacturing such microfluidic devices and to blocks of material suitable as connector.

### PRIORITY

This application claims priority to European Patent Application number 09305368.4, filed Apr. 28, 2009, titled “Microreactors with Connectors Sealed Thereon; Their Manufacturing”.

### BACKGROUND OF THE INVENTION FIELD OF THE INVENTION

The present invention concerns connection of microreactors. It more particularly relates to glass, glass-ceramic and ceramic microreactors equipped with connection systems, to a method of manufacturing the same and to blocks of material suitable as connection systems.

### TECHNICAL BACKGROUND

Microreactors (microstructures), more particularly glass, glass-ceramic and ceramic microreactors (microstructures), are described in numerous patents, for example in U.S. Pat. No. 7,007,709.

They are drilled on back or (and) front face(s) to ensure reactant(s) inlet and product(s) outlet as well as generally thermal fluid inlet and outlet. Specific connection systems have already been described.

Such connection systems have more particularly been described in patent applications FR 2 821 657 and WO 2005/107 937 (in both said prior art documents, multiport connectors with polymer seal are described. A face connection is ensured and it induces a mechanical stress on the microreactor), also in patent application EP 1 925 364 (the described connection implies the cooperation of female and male parts) and patent application US 2007/280855 (the connector is here secured to the microreactor via mechanical means (by screw, peg or other fastener)). The applicant has also proposed a specific connection system in patent application EP 1 854 543. Said specific connection system is shown in annexed prior art FIG. 1. Several connection systems 50 are present on each microreactor 20. They are shown arranged on a single face but they are generally arranged on both faces.

According to EP 1 854 543, fluidic face connection at each inlet and outlet is ensured thanks to a single port connector 50 tight on the microreactor 20 through a C-clamp mechanical part 55. Parts in contact with fluids are:

the O-ring seal 26, usually made of a perfluoroelastomer material,

the connector adapter 53 typically made of PTFE; and

the fitting 57, generally a Swagelock® fitting, usually made of PFA.

The material choices allow getting high and broad chemical resistant fluidic connection. However, internal pressure and temperature ranges of use are limited, as shown on FIG. 6. FIG. 6 actually shows in its area A the temperature and pressure operation ranges of high chemical resistance standard connection: PTFE adapter+PFA Swagelok® fitting. Stainless steel adapter and fittings would allow increasing operation conditions (higher combined pressure and temperature), but chemical compatibility would be lost for a lot of applications. Hastelloy C main issue would be its high cost, without providing so high chemical resistance.

Several microfluidic devices 200′, each comprising a microstructure 20, for example a glass microstructure, and single port connectors 50, are assembled together in a module 61, 62 of a multi-step engineered reactor 60. Such a reactor is actually able to comprise numerous modules. Reactors of that type are so able to ensure a lot of chemical reactions, especially multi-step reactions, integrating several functions like pre-heating or cooling, mixing (single injection or multi-injections), residence time . . . . Each module 61 and 62 of the reactor 60 includes three microstructures 20. The typical distance between the microstructures 20 of a module is 120 mm. Such a distance allows the face connection with the single port connectors 50.

Considering these reactors composed of several microstructures linked together using such single port connector and piping, several issues have to be considered. The first main one is limiting the connection complexity that leads to a lot of tightness locations (which are always potential sources of leakage), to long assembly and/or maintenance time, to quite large reactor footprint and significant mechanical parts cost. The second main issue to consider is the limited combined pressure and temperature operation ranges. It would be opportune to address an enlarged market with applications running at higher pressure and temperature. Some other issues may also be addressed like reducing internal volume into connection, avoiding any potential mechanical stress induced on the microstructures, proposing transparent connection zones.

The inventors have considered these numerous issues and hereafter propose a new connection concept for microreactors.

### SUMMARY OF THE INVENTION

The present invention provides a microfluidic device including a microreactor with fluidic inlet(s) and outlet(s) and a connector with fluidic channel(s) into its volume, at least one of said inlet(s) and outlet(s) of said microreactor being connected through said connector. Said microreactor is made of a first material selected from the group consisting in glasses, ceramics, glass-ceramics and metals coated with a glass, ceramic or glass-ceramic coating. Said connector is made of a second material selected from the group consisting in glasses, ceramics, glass-ceramics and metals coated with a glass, ceramic or glass-ceramics coating. Said connector is sealed on said microreactor via a fit layer made of a third material; said third material being selected from the group consisting in glasses, ceramics and glass-ceramics, having a lower softening point than the softening point of any glass, ceramic and glass-ceramic of said microreactor and connector and also having an expansion coefficient compatible with the expansion coefficient of any glass, ceramic and glass-ceramic of said microreactor and connector, (advantageously having a lower softening point than the softening points of both said first and second materials selected from glasses, ceramics and glass-ceramics or of both said coatings of said first and second metallic materials and also having an expansion coefficient compatible with the expansion coefficients of both said first and second materials selected from glasses, ceramics and glass-ceramics or of both said coatings of said first and second metallic materials).

According to some variants:

the connector is sealed on the microreactor via a frit plate (generally of a thickness e: 0.5 mm≦e≦2 mm) or via a thin layer of a frit (having generally a thickness e′: e′<500 μm);

the sealing(s) is(are) glass/glass/glass sealing(s) or ceramic/ceramic/ceramic sealing(s) or ceramic/glass/ceramic sealing(s);

the fluidic channel(s) inside the connector is(are) not straight channels, so as to create side connections. Side connections are particularly advantageous (with regards to face connections);

the connector is located on a edge of the microreactor, is advantageously located on a edge and in a corner of said microreactor;

at least two fluidic inlet(s) and outlet(s) are connected through a single connector; all fluidic inlet(s) and outlet(s) are advantageously connected through a single connector. Multiport connections are particularly advantageous;

a single connector for all fluidic inlet(s) and outlet(s) is sealed parallel to a edge of the microreactor and close to said edge, advantageously in a corner, all said inlet(s) and outlet(s) being preferably arranged on a line;

the microfluidic device is connected to a plate through a single connector arranged parallel to a edge of the microreactor and close to said edge via O-ring seals and fixed to said plate via mechanical fixing means only contacting said plate and said connector (without any mechanical contact and stress on the microreactor).

The present invention also provides a method for manufacturing such a microfluidic device. Said method comprises the sealing of at least one connector to a microreactor, said sealing being carried out during the manufacturing of said microreactor or being carried out once said microreactor has been manufactured.

According to some variants:

a sealing comprises the arrangement of a fit plate between the two surfaces to seal;

a sealing comprises the deposit of a thin layer of frit on at least one of the two surfaces to seal.

The present invention also provides a block made of a material selected from the group consisting of glasses, ceramics, glass-ceramics and metals coated with a glass, ceramic or glass-ceramic coating, having two main faces and at least a lateral one, with at least one fluidic channel through its volume, from a face to an other face, advantageously from one of its main face to a(the) lateral one, allowing fluidic connection(s), advantageously side fluidic connections. Such a block is suitable as connector for microreactors.

According to some variants:

the fluidic channel(s) has(have) an equivalent diameter within the range of 1-10 mm, advantageously within the range of 1.5-5 mm;

the block includes fluidic channels of different internal volumes within its volume;

the block includes at least one fluidic channel which separates and/or at least two fluidic channels which join together within its volume;

the block includes at least one recess for a sensor, such a recess emerging into a fluidic channel, within its volume.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention and together with the description serve to explain the principles and operations of the invention.

### BRIEF DESCRIPTION OF THE DRAWINGS

FIG. 1 (prior art) is a schematic perspective view of a multi-step engineered reactor composed of two modules including microstructures (microreactors) equipped with prior art connectors and their fixation means (shown on the enlarged detail).

FIG. 2 is a schematic perspective underside view of a multiport connector according to the invention.

FIG. 3 is a schematic perspective view of a microfluidic device of the invention: a microstructure (microreactor) equipped with its multiport connector according to the invention.

FIGS. 4A and 4B are schematic perspective views of frit plates suitable to ensure sealing between a microstructure and a multiport connector of the invention.

FIGS. 5A and 5B are schematic cross-sections (according to V-V of FIG. 3) of a sealing microstructure/connector according to the invention.

FIG. 6 shows temperature and pressure operation ranges of connectors of the invention, on the one hand and of connectors of the prior art, on the other hand.

FIG. 7 is a schematic view of an appropriate connection pattern on a microreactor able to be fitted with a multiport connector of the invention.

FIG. 8 is a schematic perspective view of an assembly including two microstructures equipped with a multiport connector according to the invention and tightened into to a plate; the fixation connector/plate being shown on an enlarged cross-section detail.

### DETAILED DESCRIPTION

Reference will now be made in detail to the presently preferred embodiments of the invention, examples of which are illustrated in the accompanying drawings. Whenever possible, the same reference numerals will be used throughout the drawings to refer to the same or like parts.

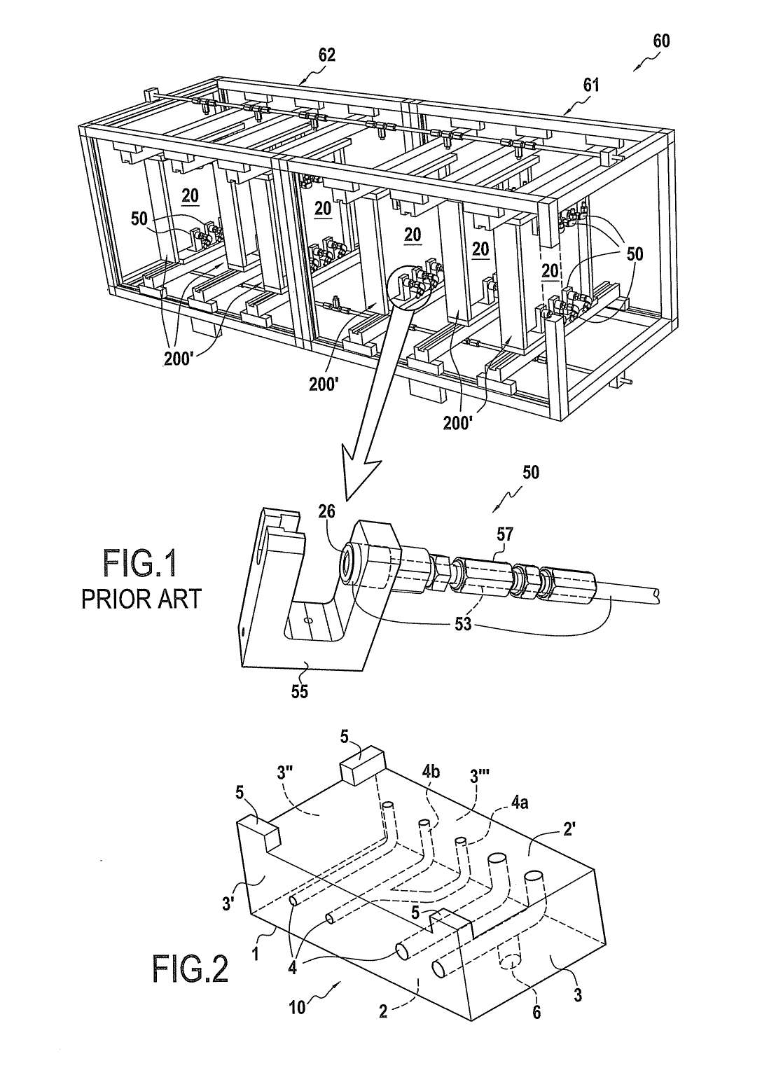

FIG. 1 (prior art) has been commented above.

FIG. 2 is a schematic perspective underside view of a multiport connector 10 of the invention. Such a connector 10 consists in a block 1 made of a glass, ceramic, glass-ceramic or metal (coated with a glass, ceramic or glass-ceramic coating) material, having two main faces 2,2′ and four lateral faces 3,3′,3″, 3′″, with fluidic channels 4 through its volume. According to a variant not shown, the block may have a cylindrical shape, with two main faces and a single lateral face.

It should be emphasized that such a block constitutes the key of the claimed invention.

The fluidic channels 4 connect the main face 2′ to a single lateral one 3′, i.e. connect two perpendicular faces, so allowing side connections. Such side connections are particularly advantageous. According to variants not shown, such channels are able to connect a main face 2, 2′ to at least two different lateral faces chosen amongst faces 3,3′,3″ and 3′″ and/or are able to connect opposite faces of a block and/or are able to connect both perpendicular and opposite faces, so allowing both side and face connections.

The block 1 of FIG. 2 allows numerous (side) connections. So it is called a multiport connector 10. The connectors or blocks of the invention are generally able to ensure 2 to 10 connections. However, it should be noted that the scope of the claimed invention also encompasses single port connectors, i.e. blocks with a single channel through their volume . . . . Multiport connectors are obviously preferred. Multiport connectors with their channels 4 arranged along a line are particularly preferred.

The diameter of the fluidic channel(s) is generally within the range of 1-10 mm, advantageously within the range of 1.5-5 mm. Said diameter is determined by the properties of the fluid intended to be circulated within said fluidic channel(s), by the specific application needed. A diameter of 1.5 mm may be suitable in contexts of low volumes without thermal control, a diameter of 5 mm may be required with fluids of high viscosity or with high flow rate inducing high pressure drop . . . .

As shown in FIG. 2, the diameters of the fluid channels 4 inside the block 1 can be different (the diameter of a channel is different from the diameter of at least another channel). Generally speaking, the fluidic channels inside a connector can have different internal volumes. It is also possible to have a fluid channel which separates inside the block in two different channels (variant not shown) and/or at least two fluid channels which join together inside the block. Said last variant is shown on FIG. 2. Fluidic channels 4a and 4b join together to have a single channel 4 emerging.

The diameter of any fluidic channel is more precisely an equivalent diameter insofar as any fluidic channel is not compulsorily cylindrical (with circular section). It is quite possible to have any channel with a non circular section, for example with a rectangular section, more particularly when the connector is obtained by hot forming.

The multiport connector 10 of the invention, as shown in FIG. 2, also presents (in its block 1 of material) a suitable recess 6 emerging into a fluidic channel 4, able to receive a sensor. Such a sensor may be used to measure the temperature of the fluid circulating inside the channel 4 and/or the flow rate of such a fluid.

The block 1 of material also comprises alignment pins 5 used to correctly position and maintain it during its sealing by heat to a microreactor (so as to constitute a microfluidic of the invention). The holes (inlet(s) and outlet(s)) of the microreactor (see FIG. 7)) have to be aligned with the outflow(s) and inflow(s) of the fluidic channels.

According to a variant not shown, the connector 10 may allow cross-connections, i.e. the block 1 may include fluidic channels which cross (so that, for example, at least one inlet and at least one outlet cross).

Characteristically, the block 1 is intended to be sealed on a microreactor 20, fitting with the inlets and outlets of said microreactor 20, so as to be able to ensure its function of connector.

Details are now given on the constitutive materials of the microreactor 20 and of the block 1, suitable as connector 10.

The microreactor 20 is made of a first material selected from the group consisting in glasses, ceramics, glass-ceramics and metals coated with a glass, ceramic or glass-ceramic coating.

The block 1 intended to be used as connector 10 after sealing (by heat) on the microreactor 20 is made of a second material also selected from the group consisting in glasses (Pyrex or Pyrex-like glasses for example), ceramics (alumina for example), glass-ceramics and metal coated with a glass, ceramic or glass-ceramic coating.

One skilled in the art knows how to choose both said first and second materials to have them resist to the circulation of fluids. He easily realizes that the chemical durability of the connector has advantageously to be at least equal to the one of the microreactor. So the second material generally shows a chemical resistance equal to or greater than the one of said first material. These notions of chemical resistance and chemical durability are familiar to those skilled in the art. They are quantified by measures of weight loss of samples. There exist normalized tests well-known from one skilled in the art (for example, test DIN 12116 for the resistance to acids and test ISO 695 for the resistance to bases). Said second material selected from glasses, ceramics and glass-ceramics or said coating of said second metallic material can advantageously have a softening point (one skilled in the art knows that parameter, he knows normalized methods to measure it, more particularly the one according to the standard ASTM C 1351M) equal to or greater than, the one of said first material selected from glasses, ceramics and glass-ceramics or of said coating of said first metallic material. In that way, during the sealing thermal cycle, there will be no risk at all of deformation of the block 1 and so connection face will be kept flat enough to get tightness with suitable polymer O-ring seals.

The block 1 can be realized by standard machining or carrying out hot forming processes.

Precisions on the way of sealing the block 1 on the microreactor 20 are given below in reference to FIGS. 4A, 4B, 5A and 5B.

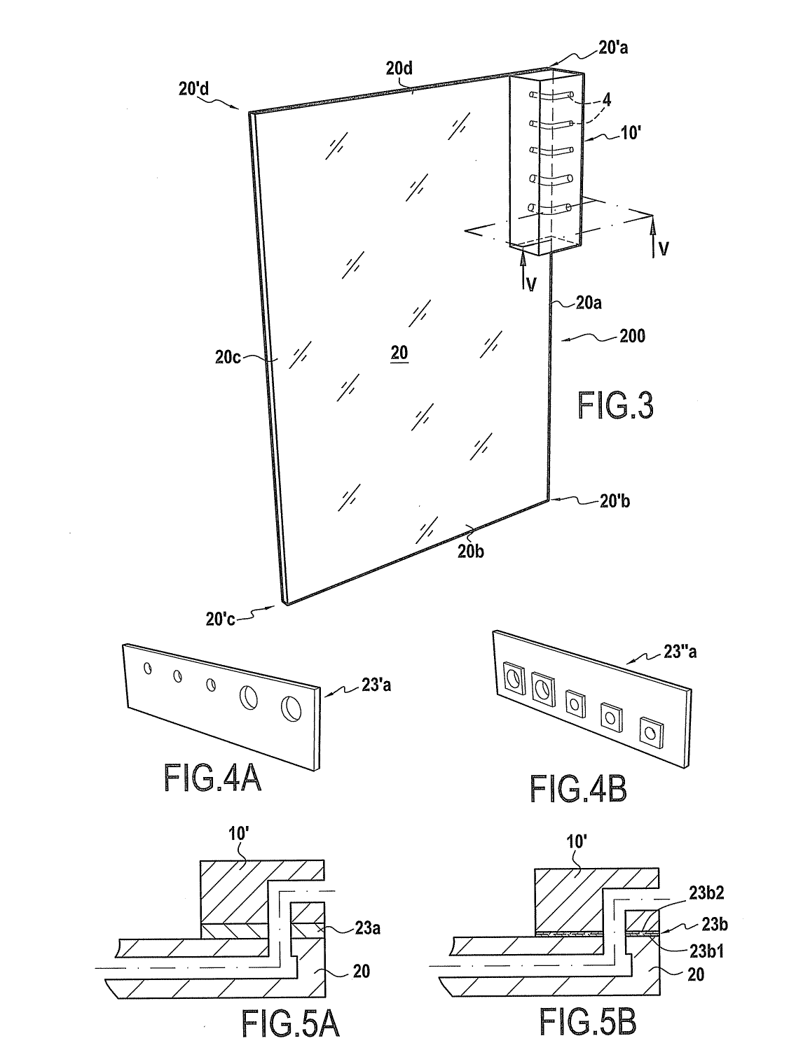

FIG. 3 shows a microfluidic device 200 comprising a microreactor 20 equipped with a single connector 10′. Said single connector 10′ allows side fluidic connections through five fluidic channels 4. None of said fluidic channels separates or joins. Said single connector 10′ has been sealed in front of the fluidic inlets and outlets of the microreactor 20 and is able to drive all said fluidic inlets and outlets (arranged on the same face of said microreactor) on a single connection face perpendicular to the microfluidic surface, using the fluidic channels 4. Said fluidic channels 4 are suitable to inject or receive reactants, products and heat exchange fluids.

The microreactor 20 has its surface delimited by four edges 20a, 20b, 20c and 20d. Edges 20a and 20b join in corner 20′b, edges 20b and 20c join in corner 20′c, edges 20c and 20d join in corner 20′d and edges 20d and 20a join in corner 20′a.

According to a preferred mode of the invention shown in FIG. 3, the single connector 10′ is sealed in front of all the fluidic inlets and outlets, arranged on a line. It is sealed parallel to the edge 20a of the microreactor 20, close to said edge 20a, in the corner 20′a.

The sealing between a microreactor 20 and a connector of the invention such as 10 on FIGS. 2, 10′ on FIGS. 3, 5A, 5B and 8 may be carried out according to different methods.

It may be carried out using a frit plate. Such a fit plate may exist according to different designs. Two designs are shown on FIGS. 4A and 4B. Such a fit plate is a precursor of the frit layer 23a shown on FIG. 5A. Such a fit plate is made of a suitable material: the third material detailed above: a glass, a ceramic or a glass-ceramic having a suitable chemical resistance, a (lower) softening point and a suitable expansion coefficient.

FIG. 4A shows a plane plate 23′ a with hole drillings. The diameter of the holes is advantageously a bit larger (+0.5 mm, generally) than the one(s) of the channels 4 of the connector 10 or 10′. So we may indicate in a non limitative manner that such holes have generally diameters from 2 mm to 5.5 mm.

FIG. 4B shows a plate 23″a with structured pad on both sides, on which same kind of holes are drilled.

Using a fit plate 23′a or 23″a for carrying out a sealing of the connector 10 on the microstructure 20 will more particularly depend on the surface quality and geometrical flatness of the microstructure 20.

Such frit plates 23′a or 23″a can be realized by standard machining or using hot forming processes.

FIG. 5A illustrates the sealing principle (using a frit plate): a lower softening point frit plate 23′a or 23″a has been used to seal two materials having higher softening point, the connector 10′ and the microreactor 20. Said connector 10′ and microreactor 20 are then sealed via the fit layer 23a. Note that the connector 10′ can be so sealed (the sealing comprising the arrangement of a frit plate between the two surfaces to seal) on a pre-constituted microreactor 20 (first variant) but that the sealing heat treatment (or thermal sintering cycle) can also be carried out to seal an assembly comprising the constitutive layers of the microreactor 20, the connector 10′ and the frit plate 23′a or 23″a (second variant). Such a sealing heat treatment is so used both to constitute the microreactor 20 and to seal the connector 10′ on its surface.

The method according to said second variant comprises:
manufacturing the constitutive parts of the microfluidic device: the constitutive layers of the microreactor 20, the connector(s) 10,10′ and the frit plate(s) 23′a, 23″a;
assembling them together; and
heat treating the assembly so as to have all said constitutive parts sealed together.

The result of the sealing is a microfluidic device, a continuous structure including the microreactor and the connector(s), able to withstand more than 40 bars. Note also that several connectors 10′ (or 10) may be sealed on a microreactor 20, keeping in mind that a single connector 10′ (or 10) used for all inlets and outlets is a preferred variant.

FIG. 5B illustrates the sealing principle according to another method, not using a frit plate as precursor of the sealing fit layer but using two thin layers of frit. According to said method, the sealing frit layer 23b is obtained from two thin layers of fit 23b1 and 23b2 deposited on the surfaces to seal. The method carried out is the following.

The microreactor 20 is preconstituted. On a part of its external surface (where the connector 10′ is intended to be sealed), a thin layer of fit 23b1 is deposited.

At least a connector 10′ is also preconstituted and a thin layer of frit 23b2 is deposited on a part of its external surface, said part being intended to be sealed on said microreactor 20. The thin layer of fit 23b2 is generally deposited on a face of the connector 10, taking care of not blocking the fluidic channel(s) 4.

The two deposited thin layers of frit 23b1 and 23b2 are then contacted and following a suitable heat treatment, they generate the seal or fit layer 23b.

Said method thus comprises:
manufacturing a microreactor 20 and depositing a thin layer of frit 23b1 on a part of its external surface;
manufacturing at least a connector 10,10′ and depositing a thin layer of fit 23b2 on a part of its external surface, said part being intended to be sealed on said microreactor 20,
contacting said deposited two thin layers of frit 23b1 and 23b2,
heat treating the so constituted assembly so as to have said two layers of fit 23b1 and 23b2 sealed.

It is quite possible to obtain a good sealing using a single thin layer of frit deposited on one of the two surfaces to seal. So the present disclosed method includes both the use of a single and of two thin layers of frit.

It is also quite possible to carry out that method of sealing—using a single or two thin layers of fit hereabove described carried out on a pre-constituted microreactor, while constituting the microreactor (a thin layer of frit (the single one thin layer of frit or one of the two thin layers of fit intended to form the frit layer of the final microfluidic device) being deposited on a part of the external surface of a suitable constitutive layer of the microreactor).

It has been indicated that a fit layer 23a obtained from a fit plate 23′a or 23″a has generally a thickness comprised between 0.5 and 2 mm while a frit layer 23b (obtained from one or two thin layers 23b1 and 23b2) has generally a thickness equal or inferior to 500 μm.

Whatever the exact variant of sealing carried out, it is advantageous to seal materials of the same kind. So the microfluidic devices of the invention comprise advantageously a glass microreactor with glass connector(s), a ceramic microreactor with ceramic connector(s), or a glass-ceramic microreactor with glass-ceramic connector(s).

The microfluidic devices of the invention are very advantageously glass microreactors with glass connectors or ceramic microreactors with ceramic connectors. The sealings are obviously carried out with suitable frit material. So the preferred sealings microreactor/frit layer/connector(s) are glass/glass/glass sealings, ceramic/ceramic/ceramic sealings and ceramic/glass/ceramic sealings. A microfluidic device of the invention comprises a sealing microreactor/frit layer/connector or at least two sealings microreactor/frit layers/connectors.

Concerning the material of the frit layer (the third material), it has to show a suitable softening point and a suitable expansion coefficient (to be able to constitute an effective seal between the first and second material). Its softening point has to be lower than the softening point of any glass, ceramic and glass-ceramic of the microreactor and connector and its expansion coefficient has to be compatible with the expansion coefficient of any glass, ceramic and glass-ceramic of the microreactor and the connector (said microreactor and connector being made of these materials (glass, ceramic, glass-ceramic) or including these materials as coating of metal). Advantageously, said third material has a lower softening point than the softening points of both said first and second materials selected from glasses, ceramics and glass-ceramics or of both said coatings of said first and second metallic materials and also has an expansion coefficient compatible with the expansion coefficients of both said first and second materials selected from glasses, ceramics and glass-ceramics or of both said coatings of said first and second metallic materials. In reference to said expansion coefficient of the third material, it is suitable (“compatible”) if its value differs from the values of the expansion coefficients of both the first and second materials of less than 20×10⁻⁷K⁻¹, advantageously less than 10×10⁻⁷ K⁻¹ (all these CTE values being considered between 25 and 300° C., being expressed in 10⁻⁷ K⁻¹).

One skilled in the art also knows how to choose said third material to have it resist to the circulation of fluids. He easily realizes that the chemical durability of said third material, as the one of the second material, has advantageously to be at least equal to the one of the first material. So said third material generally shows a chemical resistance equal or greater than the one of said first material.

We remind here that a single multiport connector is advantageously used.

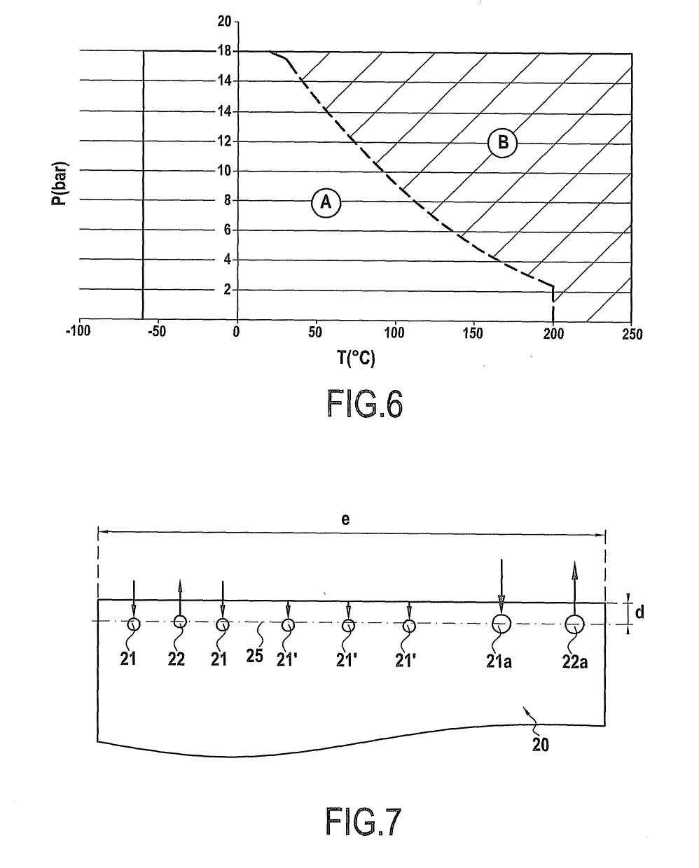

The main advantage of the invention connection concept is visualized on FIG. 6.

We have indicated that connections according to the prior art (EP 1 854 543—FIG. 1) are limited in terms of pressure and temperature operation ranges because of use of PTFE adapter and PFA Swagelok® fittings. These two materials are providing very high chemical compatibility but can not withstand high pressure when temperature is increasing (not higher than 10 bars at 100° C., with safety factor). O-rings seals like Chemraz® O-ring seals are not limiting factor, being able to withstand 20 bars at 250° C.

A connector according to the invention sealed on a microstructure is a concept that suppresses as well as PTFE adapter and PFA Swagelok® fitting, the two limiting components. The single remaining component is the O-ring seal.

In consequence, the acceptable combined pressure and temperature operation ranges are increasing, towards 20 bars up to 250° C. and therefore covering enlarged chemical applications.

FIG. 6 shows the limited operating conditions of high chemical resistant prior art (EP 1 854 543—FIG. 1) connections: area A and the enlarged operating conditions of the connections according to the invention: areas A+B.

It has already been mentioned that a single port connector may be sealed according to the invention to an inlet or outlet of a microreactor but that multiport connectors are obviously preferred, that such multiport connectors are advantageously arranged on an edge of the microreactor, close to said edge, with all the inlet(s) and outlet(s) of said microreactor very advantageously arranged on a line. Such a design of a microreactor is illustrated in FIG. 7.

In any way, the pattern of the microreactor and the one of the connector have obviously to be adapted (to match) to allow the connection(s).

According to the preferred variant shown in FIG. 7, all the fluidic inlets 21, 21′, 21a and outlets 22, 22a are located on a line 25 parallel to an edge of the microstructure 20 and close to said edge, also close enough to limit size of the multiport connector (to seal in front).

According to the illustrated variant, 21 are inlets for different reactants, 21a is the inlet for the heat exchange fluid while 21′ are additional potential injection points; 22 is the outlet for the product(s) and 22a is the outlet for the heat exchange fluid.

Typical distance d (between the line 25 and the edge of the microfluidic device 20) is comprised between 5 and 30 mm, while typical distance e (which represents the length of a suitable connector to seal) is comprised between 20 and 150 mm. We have already indicated (in a non limitative way) that connectors of the invention are more particularly suitable to ensure 2 to 10 connections. So the number of fluidic inlets and outlets located in the area of the surface of the microfluidic device shown in FIG. 7 is typically from 2 to 10. All these given figures define a recommended (but not limitative) connection pattern used for the design of the fluidic microstructures of the invention. Once again, the concept of the invention may exist according to different variants, such as one using single port connectors or at least one multiport connector or a multiport connector suitable to connect inlet(s) and outlet(s) not arranged on a single line.

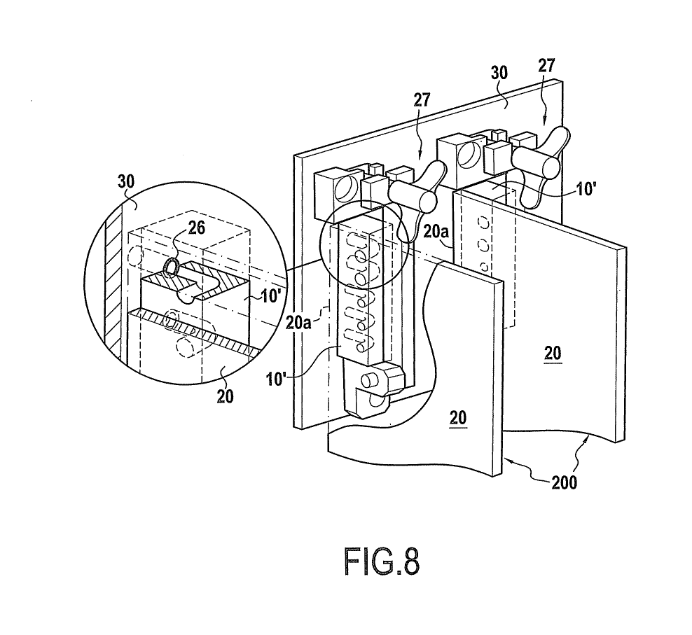

FIG. 8 shows two microfluidic devices 200 of the invention, each comprising a microreactor 20 and its multiport connector 10′ sealed thereon. Said two microfluidic devices 200 are connected to a plate 30 via their multiport connector 10′. The tightened connection plate 30/connector 10′ is ensured thanks to the O-ring seals 26 and the clamping system 27. The channels inside the thickness of the plate 30 are not shown.

It has to be emphasized that the multiport side connection according to the invention, as more particularly illustrated in this FIG. 7, is particularly advantageous:

it involves a single side connection face tightened thanks to a single clamping system 27, without any mechanical contact (stress) on the microstructure 20;

it allows the arrangement of several microfluidic devices 200 in a limited space. The distance between the microstructures 200 can be limited, can be lower than 100 mm. Said distance value has to be compared with the prior art distance of 120 mm (see FIG. 1);

it offers the possibility to design a reactor architecture based on fluidic backbone approach. Several microfluidic devices 200 can be plugged into a fluidic backbone like electronic cards, fluid communication between microstructures 20 being done through the fluidic backbone.

No doubt that one skilled in the art has realized the great interest of the invention, more particularly the great interest of the advantageous variant of the invention using a connector able to drive all the inlets and outlets on a single connection face, perpendicular to the microreactor surface.

We hereafter insist on the main advantages of the new fluidic connection approach of the invention. Some of said advantages are common to all variants, some of them are limited to specific (preferred) variants. Most of them have already been explained in reference to at least one of the accompanying figures. Most of them are hereafter explained in reference to the teaching of EP 1 854 543.

1) Large Temperature and Pressure Operation Ranges

Said operation ranges are larger than the one of the prior art connections according to EP 1 854 543 (see FIG. 6 and the corresponding above comments).

2) Simplification of the Microfluidic Device Construction: Less Clamping Systems and Tightness Zones

Proposed multiport connector sealed on a microstructure allows simplifying the mechanical structure of the microfluidic device:

instead of having one clamping system per single port connector, so per inlet and outlet, a single global clamping system for the whole multiport connector is enough. So typically five C-clamps (55 in FIG. 1) are replaced by a single system (27 in FIG. 8), which has positive impact on assembly time and mechanical parts cost,

sealing of the connector on the microreactor is a way to avoid the use of polymer sealing zone, with the associated risk of leakage.

In the case of prior art connections as described in EP 1 854 543 (see FIG. 1), making fluidic communication between two microreactors requires at least two O-ring seals and one Swagelok® fitting. According to the invention, with multiport sealed connector, in the case of a fluidic backbone approach only two O-ring seals are required. Swagelok® fitting is no more required. It removes from the system the limiting component as explained above.

3) Side Connections: Reactor Compactness and Compatibility with Fluidic Bus Reactor Architecture

Prior art connections (according to EP 1 854 543) are face connections, with single port connectors, on both sides of the microreactor (see FIG. 1). It results, as already indicated, a large reactor footprint when several microreactors are assembled together, because of the required minimum distance of 120 mm. Side connections according to the invention, which may optimized to have a single connection face perpendicular to the microreactor surface, located on a edge of the microreactor, allow a limited distance between two microreactors: ≦100 mm. For a typical structure including twelve reactors, the benefit is a footprint reduction of about 20%. As already explained in reference to FIG. 8, side connection offers the possibility of designing structures based on fluidic backbone approach . . . .

4) Low Internal Volume without Thermal Control

Typical single port connectors presented on FIG. 1, with PTFE adapter and PFA Swagelok® fitting, have an internal volume of 0.5 ml. To avoid any risk of uncontrolled reactions into connection and piping, limiting this internal volume without any thermal control is critical. Typical side connections according to the invention, as shown in the attached figures, can have an internal volume of only 0.1 ml, per connection channel.

5) Ease of Maintenance

Another benefit of sealed multiport connectors, advantageously with side connections, is the ease of plug and play. Because of the single clamping system and because of no direct contact with adjacent microstructures, it is possible to rapidly remove and exchange one microstructure of an assembly without moving the others.

With prior art connections according to EP 1 854 543, mainly based on Swagelok® fitting, an operation is needed for each single port connector and it is necessary sometimes to move several microstructures in order to remove easily one.

6) Robustness

With connections as described in EP 1 854 543, tightening of single port connectors is done into the microstructure itself, where additional stresses like internal pressure and thermal gradient have to be handled. And beyond compression stresses, potential bending stresses could be applied on microstructures when connection between microstructures is done and when piping is added, especially heat exchange stainless steel piping.

According to the invention, the single tightening force is applied only on the connector sealed on microstructure: no mechanical force is applied on the microstructure itself (even no mechanical contact needed) which contributes to increase mechanical robustness of glass microstructure. (See FIG. 8).

7) Transparent Connection

The connectors of the invention, made of a glass may be transparent. So it is possible to have visual contact of the reactant(s) and product(s), even inside connection zones (which is not possible according to prior art single port connector of EP 1 854 543). Interest is to detect any potential clogging into inlets and outlets zones. So the advantage of the transparency of a microreactor may be kept into a connector of the invention.

The microfluidic devices disclosed herein are generally useful in performing any process that involves mixing, separation, extraction, crystallization, precipitation, or otherwise processing fluids or mixtures of fluids, including multiphase mixtures of fluids—and including fluids or mixtures of fluids including multiphase mixtures of fluids that also contain solids—within a microstructure. The processing may include a physical process, a chemical reaction defined as a process that results in the interconversion of organic, inorganic, or both organic and inorganic species, a biochemical process, or any other form of processing. The following non-limiting list of reactions may be performed within the disclosed devices: oxidation; reduction; substitution; elimination; addition; ligand exchange; metal exchange; and ion exchange. More specifically, reactions of any of the following non-limiting list may be performed within the disclosed devices: polymerisation; alkylation; dealkylation; nitration; peroxidation; sulfoxidation; epoxidation; ammoxidation; hydrogenation; dehydrogenation; organometallic reactions; precious metal chemistry/homogeneous catalyst reactions; carbonylation; thiocarbonylation; alkoxylation; halogenation; dehydrohalogenation; dehalogenation; hydroformylation; carboxylation; decarboxylation; amination; arylation; peptide coupling; aldol condensation; cyclocondensation; dehydrocyclization; esterification; amination; heterocyclic synthesis; dehydration; alcoholysis; hydrolysis; ammonolysis; etherification; enzymatic synthesis; ketalization; saponification; isomerisation; quaternization; formylation; phase transfer reactions; silylations; nitrile synthesis; phosphorylation; ozonolysis; azide chemistry; metathesis; hydrosilylation; coupling reactions; and enzymatic reactions.

1. A microfluidic device including a microreactor with fluidic inlet(s) and outlet(s) and a connector with fluidic channel(s) into its volume, at least one of said inlet(s) and outlet(s) of said microreactor being connected through said connector characterized in that:
said microreactor is made of a first material selected from the group consisting in glasses, ceramics, glass-ceramics and metals coated with a glass, ceramic or glass-ceramic coating;
said connector is made of a second material selected from the group consisting in glasses, ceramics, glass-ceramics and metals coated with a glass, ceramic or glass-ceramics coating; and
said connector is sealed on said microreactor via a frit layer made of a third material; said third material being selected from the group consisting in glasses, ceramics and glass-ceramics, having a lower softening point than the softening point of any glass, ceramic and glass-ceramic of said microreactor and connector and also having an expansion coefficient compatible with the expansion coefficient of any glass, ceramic and glass-ceramic of said microreactor and connector.

2. The microfluidic device according to claim 1, characterized in that said third material has a lower softening point than the softening points of both said first and second materials selected from glasses, ceramics and glass-ceramics or of both said coatings of said first and second metallic materials and also has an expansion coefficient compatible with the expansion coefficients of both said first and second materials selected from glasses, ceramics and glass-ceramics or of both said coatings of said first and second metallic materials.

3. The microfluidic device according to claim 1, wherein said connector is sealed on said microreactor via a frit plate or via a thin layer of a frit.

4. The microfluidic device according to claim 1, wherein the sealing(s) is(are) glass/glass/glass sealing(s), ceramic/ceramic/ceramic or ceramic/glass/ceramic sealing(s).

5. The microfluidic device according to claim 1, wherein the fluidic channel(s) inside the connector is(are) not straight channels, so as to create side connections.

6. The microfluidic device according to claim 1, wherein said connector (10′) is located on a edge of said microreactor, is advantageously located on a edge and in a corner of said microreactor.

7. The microfluidic device according to claim 1, wherein at least two fluidic inlet(s) and outlet(s) are connected through a single connector, wherein all fluidic inlet(s) (21; 21a) and outlet(s) are advantageously connected through a single connector.

8. The microfluidic device (200) according to claim 1, wherein a single connector for all fluidic inlet(s) and outlet(s) is sealed parallel to a edge of said microreactor and close to said edge, advantageously in a corner, all said inlet(s) and outlet(s) being preferably arranged on a line.

9. The microfluidic device according to claim 1, wherein it is connected to a plate through a single connector arranged parallel to a edge of said microreactor and close to said edge via o-ring seals and fixed to said plate via mechanical fixing means only contacting said plate and said connector.

10. A method for manufacturing a microfluidic device according to claim 1, wherein said method comprises the sealing of at least one connector to a microreactor, said sealing being carried out during the manufacturing of said microreactor (20) or being carried out once said microreactor has been manufactured.

11. The method according to claim 10, wherein said sealing comprises the arrangement of a frit plate between the two surfaces to seal.

12. The method according to claim 10, wherein said sealing comprises the deposit of a thin layer of frit on at least one of the two surfaces to seal.

13. A block made of a material selected from the group consisting of glasses, ceramics, glass-ceramics and metals coated with a glass, ceramic or glass-ceramic coating, having two main faces and at least a lateral one, with at least one fluidic channel through its volume, from a face to an other face, advantageously from one of its main face to a(the) lateral one, allowing fluidic connection(s), advantageously side fluidic connections.

14. The block according to claim 13, wherein the fluidic channel(s) has(have) an equivalent diameter within the range of 1-10 mm, advantageously within the range of 1.5-5 mm.

15. The block according to claim 13, wherein its volume includes fluidic channels of different internal volumes.

16. The block according to claim 13, wherein its volume includes at least one fluidic channel which separates and/or at least two fluidic channels which join together.

17. The block according to claim 13, wherein its volume also includes at least one recess for a sensor, such a recess emerging into a fluidic channel.
